(19) United States
(12) Patent Application Publication
(10) Pub. No.: US 2010/0189675 A1
(43) Pub. Date: Jul. 29, 2010

(54) COSMETIC USE OF AN IMIDOPERCARBOXYLIC ACID DERIVATIVE AS DESQUAMATING AGENT

(75) Inventor: Pascale Pelletier, Anthony (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'Oreal, Paris (FR)

(21) Appl. No.: 12/666,466

(22) PCT Filed: Jun. 24, 2008
(86) PCT No.: PCT/IB08/52507

§ 371 Date: Mar. 30, 2010

Related U.S. Application Data
(60) Provisional application No. 60/981,552, filed on Oct. 22, 2007.

(30) Foreign Application Priority Data
Jun. 26, 2007 (FR) 0756026

(51) Int. Cl. A61K 8/49; A61K 8/98; A61K 36/03; A61K 31/7008; A61K 31/496; A61Q 19/00; A61Q 19/08

(52) U.S. Cl. 424/70.1; 424/537; 424/195.17; 424/769; 514/62; 514/252.12; 514/425

(57) ABSTRACT

The present invention relates to the cosmetic use of at least one imidopercarboxylic acid derivative in a cosmetic composition comprising a physiologically acceptable medium, as agent for promoting desquamation of the skin and/or the scalp and/or for stimulating epidermal renewal, the associated method of cosmetic treatment and a composition comprising the said imidopercarboxylic acid derivative and a desquamating agent and/or an anti-ageing agent.

This non provisional application claims the benefit of French Application No. 07 56026 filed on Jun. 26, 2007 and U.S. Provisional Application No. 60/981,552 filed on Oct. 22, 2007.

The present invention relates to the cosmetic use of at least one imidopercarboxylic acid derivative in a cosmetic composition comprising a physiologically acceptable medium, as agent for promoting desquamation of the skin and/or the scalp and/or for stimulating epidermal renewal.

For the purposes of the invention, the expression “skin” is understood to mean, in a broad sense, the skin and the semimucous membranes (lips).

Desquamation is a natural phenomenon linked to the fact that the epidermis, which constitutes the top layer of the skin and of the scalp, is constantly regenerated.

The epidermis consists of several strata of cells, of which the deepest is the basal stratum consisting of undifferentiated cells. Over the course of time, these cells will differentiate and migrate to the surface of the epidermis, thereby constituting the various strata of the latter, as far as forming, at the surface of the epidermis, the corneocytes which are dead cells which are removed by desquamation. This surface loss is compensated by the migration of cells of the basal stratum to the surface of the epidermis. This constitutes the perpetual renewal of the skin. A forced elimination of the horny layer accelerates the renewal and makes it possible to combat ageing.

At the same time, these cells continue their differentiation, the last stage of which is the corneocyte. It in fact consists of dead cells which constitute the last layer of the epidermis, that is to say the outermost layer also called stratum corneum.

It is known that the desquamation process may be adversely affected by exogenous factors (for example: UV radiation, pollution, allergenic agents, pathogens), and/or endogenous factors (for example: hormonal changes, age . . . ) and may lead in particular to a slowing of epidermal renewal and consequently ageing of the skin, and/or thickening of the horny layer (for example: formation of callosities), and/or desquamative disorders of the aesthetic type (for example: dandruff, squamae . . . ) or of the pathological type (for example: xerosis, ichthyosis, psoriasis, atopic dermatitis).

As regards more particularly skin ageing, resulting from intrinsic or extrinsic factors, it generally results in the appearance of wrinkles and fine lines, in yellowing of the skin which develops a wrinkled appearance accompanied by the appearance of pigmented spots, in the disorganization of the elastin and collagen fibres, causing a loss of elasticity, suppleness and firmness, or in the appearance of telangiectasias.

Some of these signs of ageing are more particularly linked to intrinsic or physiological ageing, that is to say to age-related “normal” or chronobiological ageing, while others are more specific to extrinsic ageing, that is to say ageing caused in a general manner by the environment; it is more particularly photoageing due to exposure to the sun, to light or to any other radiation.

The skin changes due to intrinsic ageing are the consequence of a genetically programmed senescence in which endogenous factors are involved. This intrinsic ageing causes in particular slowing of the renewal of skin cells, which results mainly in the appearance of clinical impairments such as the reduction in the subcutaneous adipose tissue and the appearance of fine wrinkles or fine lines, and in histopathological changes such as an increase in the number and the thickness of elastic fibres, a loss in vertical fibres of the membrane of the elastic tissue, and the presence of large irregular fibroblasts in the cells of this elastic tissue.

By contrast, extrinsic ageing causes clinical impairments such as thick wrinkles and the formation of a soft and weather-beaten skin, and histopathological changes such as an excessive accumulation of elastic material in the top dermis and degeneration of the collagen fibres.

The invention relates in particular to intrinsic or physiological ageing as well as extrinsic ageing.

Various agents intended for combating skin ageing are known in the prior art, among which are antioxidants, agents promoting proliferation and/or differentiation of dermal and/or epidermal cells, agents stimulating the expression of dermal or epidermal macromolecules, that is as many agents as cellular or molecular targets in the skin for preventing and/or reducing the multiple effects of skin ageing.

Accordingly, U.S. Pat. No. 4,603,146 describes the use of retinoic acid and its derivatives in cosmetic compositions for combating skin ageing. In addition to these agents intended for combating skin ageing are also desquamating agents. In general, desquamating agents act by facilitating the elimination of the dead cells located at the surface of the horny layer of the epidermis. This “desquamating” property is also called, often wrongly, keratolytic properties.

Among these cosmetic agents promoting desquamation, that is to say the elimination of “dead” cells located at the surface of the horny layer of the epidermis, there may be mentioned in particular α-hydroxy acids (AHA) such as lactic acid or glycolic acid or β-hydroxy acids (BHA) such as salicylic acid. These active agents induce, upon topical application at concentrations of a few percentages, visible desquamation after a few days. Unfortunately, some desquamating compounds can have side effects, such as skin discomfort or even irritation.

A need therefore exists to find novel prodesquamating compounds which do not have the side effects mentioned above.

The problem which the present invention aims to solve moreover consists in providing novel methods which make it possible to maintain or improve the appearance of the skin.

The inventors have discovered, surprisingly, that some imidopercarboxylic acid derivatives possess a desquamating action on the skin and/or on the scalp.

Some of these derivatives are in particular described in the document EP 325 289.

They are commonly used as detergent in bleaching compositions, in particular for textiles.

A method is moreover known from the document U.S. Pat. No. 6,309,654 for improving microcirculation in the skin, comprising the topical application of a pharmaceutical composition containing an activated oxygen-containing compound, including a peroxycarboxylic acid. Some peroxycarboxylic acids are also known, for example from the document WO 96/05802, to be capable of being used in oral compositions for whitening the teeth.

In addition, the document EP 895 777 describes compositions, including cosmetic compositions, for example in cream form, comprising phthalimidopercarboxylic acid in the form of an adduct with cyclodextrin.

Accordingly, according to one of its aspects, the invention relates to the cosmetic use of at least one imidopercarboxylic derivative in a cosmetic composition comprising a physiologically acceptable medium, as cosmetic agent intended for promoting desquamation of the skin and/or the scalp and/or for stimulating epidermal renewal.

According to a first embodiment, the composition is intended for preventing, attenuating and/or combating the signs of skin ageing.

According to another embodiment, the composition is intended for improving the appearance and/or the texture of the skin and/or of the scalp.

The said composition may also be intended for improving the radiance and the homogeneity of the complexion and/or for smoothing the skin.

According to a particular embodiment, the said composition is intended for reducing the surface irregularities and the skin microrelief.

According to yet another embodiment, the said composition is intended for promoting the elimination of dead cells at the surface of the body.

It is also intended for promoting the elimination of dandruff.

According to another embodiment, the composition is intended for improving the staying power of the make-up.

It may also be intended for improving the result of skin treatment with colorants for the stratum corneum such as dihydroxyacetone.

According to another of its aspects, the subject of the invention is a cosmetic and/or dermatological composition comprising, in a physiologically acceptable medium, at least one imidopercarboxylic derivative and at least one auxiliary desquamating agent in particular chosen from beta-hydroxy acids such as salicylic acid and its derivatives, in particular 5-n-octanoylsalicylic acid, urea and its derivatives chosen from hydroxyethylurea, in particular N-(2-hydroxyethyl)urea marketed under the name Hydrovance® by National Starch, a thiourea, N-2-hydroxyethylpiperazine-N-2-ethanesulphonic acid (HEPES), Sophora japonica extract, honey, N-acetylglucosamine, 2-oxothiazolidine-4-carboxylic acid (procysteine) derivatives, extracts of laminaria such as laminaria saccharina and laminaria ochroleuca and mixtures thereof.

According to yet another of its aspects, the subject of the invention is a cosmetic and/or dermatological composition comprising, in a physiologically acceptable medium, at least one imidopercarboxylic derivative and at least one anti-ageing agent in particular chosen from an antiglycation agent, an NO-synthase inhibitor, an agent acting on dermal or epidermal macromolecules and/or preventing their degradation, an agent modulating the differentiation or the proliferation of cells, and in particular the differentiation of the keratinocytes and/or the proliferation of the fibroblasts or of the keratinocytes and a muscle relaxant and/or a skin relaxant.

The subject of the invention is also a method of cosmetic treatment in particular for non-therapeutic care of the skin and/or the scalp, intended for promoting desquamation of the skin and/or of the scalp and/or for stimulating epidermal renewal, characterized in that it comprises the application, to the skin and/or the scalp, of a composition comprising at least one imidoperoxycarboxylic acid derivative, in particular a compound of formula (I) or (II) as defined below.

The invention also relates to a peeling method.

More particularly, the invention relates to a method for the cosmetic treatment of the visible and/or tactile irregularities of the human skin, comprising the steps consisting in:

a) topically applying to the skin a composition comprising at least one imidopercarboxylic acid derivative,

b) leaving the composition in contact with the skin for an appropriate period for the pro-desquamating action of the said derivative, which may be between 5 min and 6 hours, preferably between 5 min and 30 min, and

c) eliminating the composition by rinsing.

Whether it is the method of cosmetic treatment for the non-therapeutic care of the skin and/or of the scalp, or the peeling method, which is understood in the context of the present invention to mean “method in accordance with the invention”, it is most particularly intended for the cosmetic treatment of aged skin and/or of dry skin.

The method in accordance with the invention is additionally most particularly intended for at least one of the conditions chosen from: preventing, attenuating and/or combating the signs of skin ageing, and in particular attenuating wrinkles and fine lines, improving the appearance and/or the texture of the skin, in particular improving the homogeneity of the complexion and/or smoothing the skin, and in particular attenuating acne or chickenpox marks and/or clearing the skin pores.

The composition for carrying out the method of cosmetic treatment for the non-therapeutic care of the skin and/or of the scalp or the cosmetic use according to the invention, or in other words the use or the method in accordance with the invention is more particularly intended for promoting desquamation of the skin and/or of the scalp, and/or for stimulating epidermal renewal, and in particular for treating at least one of the conditions chosen from:
preventing, attenuating and/or combating the signs of skin ageing,
improving the appearance and/or the texture of the skin and/or of the scalp,
improving the radiance and the homogeneity of the complexion and/or smoothing the skin,
reducing the surface irregularities and the skin microrelief,
promoting the elimination of dead cells at the surface of the body,
promoting the elimination of dandruff,
improving the staying power of the makeup, and
improving the result of treatment of the skin with colorants for the stratum corneum such as dihydroxyacetone (DHA).

According to one embodiment, the invention relates to a method of cosmetic treatment, in particular for the non-therapeutic care, of the skin, intended for preventing, attenuating and/or combating the signs of skin ageing, characterized in that it comprises the application, to the skin, of a composition comprising at least one imidoperoxycarboxylic acid derivative, in particular a compound of formula (I) or (II) as defined below.

According to another embodiment, the invention relates to a method of cosmetic treatment, in particular for the non-therapeutic care, of the skin and/or of the scalp, in particular of aged skin and/or of dry skin, intended for improving the appearance and/or the structure of the skin and/or of the scalp, characterized in that it comprises the application, to the said skin and/or the said scalp, of a composition comprising at least one imidoperoxycarboxylic acid derivative, in particular a compound of formula (I) or (II) as defined below.

According to yet another embodiment, the invention relates to a method of cosmetic treatment, in particular for the non-therapeutic care of the skin, in particular of aged skin and/or of dry skin, intended for improving the radiance and the homogeneity of the complexion and/or for smoothing the skin, characterized in that it comprises the application, to the said skin, of a composition comprising at least one imidoperoxycarboxylic acid derivative, in particular a compound of formula (I) or (II) as defined below.

According to yet another embodiment, the invention relates to a method of cosmetic treatment, in particular for the non-therapeutic care of the skin, in particular of aged skin and/or of dry skin, intended for reducing the surface irregularities and the skin microrelief, characterized in that it comprises the application, to the said skin, of a composition comprising at least one imidoperoxycarboxylic acid derivative, in particular a compound of formula (I) or (II) as defined below.

According to yet another embodiment, the invention relates to a method of cosmetic treatment, in particular for the non-therapeutic care of the skin and/or of the scalp, in particular of aged skin and/or of dry skin, intended for promoting the elimination of dead cells at the surface of the body, characterized in that it comprises the application, to the said skin and/or the said scalp, of a composition comprising at least one imidoperoxycarboxylic acid derivative, in particular a compound of formula (I) or (II) as defined below.

According to yet another embodiment, the invention relates to a method of cosmetic treatment, in particular for the non-therapeutic care of the scalp, intended for promoting the elimination of dandruff, characterized in that it comprises the application, to the scalp, of a composition comprising at least one imidoperoxycarboxylic acid derivative, in particular a compound of formula (I) or (II) as defined below.

According to yet another embodiment, the invention relates to a method of cosmetic treatment, in particular for the non-therapeutic care of the skin, intended for improving the staying power of the make-up and/or for improving the result of treatment with colorants for the stratum corneum such as dihydroxyacetone (DHA), characterized in that it comprises the application, to the skin, of a composition comprising at least one imidoperoxycarboxylic acid derivative, in particular a compound of formula (I) or (II) as defined below.

The improvement in the staying power of the make-up and/or in the treatments for colouring the skin with DHA results from the fact that the skin and/or the scalp may be prepared before the abovementioned making up and/or colouring by applying a composition according to the invention, which will promote the result of a simultaneous or subsequent treatment with a make-up agent or a colouring agent.

The expression “sign of skin ageing” is understood to mean any modification of the external appearance of the skin and/or its texture due to chronological or photoinduced ageing such as, for example, wrinkles, fine lines, withered skin, flabby skin, skin of reduced thickness, lack of skin elasticity or tone.

The prodesquamating activity is indeed mainly desired in the field of so-called anti-ageing compositions, namely compositions intended for combating the skin signs due to ageing and/or photoageing. However, the combination used according to the present invention may also find applications in the attenuation of the surface irregularities of the skin and the improvement of the skin microrelief, in particular the attenuation of acne or chickenpox marks, the clearing of the skin pores and the treatment of dry skin.

To carry out the invention, the aromatic imidopercarboxylic acid derivative may be applied to any region of the skin or of its annexes, in particular of the face, of the neck and shoulders, of the hands, or on the lips, in order to attenuate the visible and/or tactile irregularities of the skin, for example in order to attenuate scars, in order to smooth the surface and/or eliminate the dead skins in particular of the lips.

The expression “acne or chickenpox marks” is understood to mean, in the context of the present invention, the sequellae and/or scars left by these two pathological conditions in terms more particularly of skin microrelief. In other words, the treatment of the conditions in themselves, acne and chickenpox, is not covered by the present invention.

In addition, numerous skin pathologies are characterized by the production of a thick horny layer and by an abnormal desquamation, that is to say by hyperkeratosis. The latter can occur on any anatomical skin region or in a wide variety of clinical situations. Its physiopathological substratum and its cause are varied.

Advantageously, the imidopercarboxylic acid derivative makes it possible to promote desquamation of the skin and/or to stimulate epidermal renewal and therefore more particularly to treat skin and/or scalp pathologies linked to a production of a thickened horny layer and/or linked to an abnormal desquamation.

Consequently, the present invention also relates, according to another of its aspects, to the use of at least one imidopercarboxylic acid derivative for the preparation of a dermatological composition intended for treating skin and/or scalp pathologies which are characterized by the production of a thickened horny layer and/or by an abnormal desquamation.

By way of a non-limiting illustration of these skin and/or scalp disorders linked to deregulation of desquamation, there may be mentioned, in the context of the present invention:
xerosis,
hyperkeratosis,
psoriasis,
atopic dermatitis, and
ichthyosis.

The imidopercarboxylic acid derivative according to the invention makes it possible to promote desquamation of the skin and/or of the scalp and/or to stimulate epidermal renewal and may therefore be used for the preparation of a dermatological composition intended for treating the abovementioned skin pathologies which are characterized by the production of a thickened horny layer and/or by abnormal desquamation.

More particularly, the imidopercarboxylic acid derivative according to the invention makes it possible to treat the callosities or hyperkeratosis of dry feet.

Finally, the imidopercarboxylic acid derivative finds another application in the field of chemical peelings.

Peelings are a well-known means for improving the appearance and/or the texture of the skin and/or the scalp, in particular for improving the radiance and the homogeneity of the complexion and/or for reducing the visible and/or tactile irregularities of the skin, and in particular for improving the surface appearance of the skin, for attenuating acne or chickenpox marks, and for preventing, attenuating or combating the signs of skin ageing, and in particular for smoothing the irregularities of the skin texture, such as wrinkles and fine lines.

They have the effect of removing a superficial part of the skin to be treated (epidermis and optionally superficial layer of the dermis), by chemical methods.

The invention therefore relates to a peeling composition comprising, in a physiologically acceptable medium, at least one aromatic imidopercarboxylic acid derivative and in particular a compound of formula (I) or (II), as defined below and in accordance with the present invention, in an amount greater than 10% by weight of active material relative to the total weight of the composition, in particular ranging from 11% to 60% by weight of active material, in particular from 11 to 40% and more preferably still from 11 to 30% by weight of active material, relative to the total weight of the composition.

The subject of the invention is also a method for the treatment, in particular the cosmetic treatment, of the visible and/or tactile irregularities of the human skin, comprising the steps consisting in:

a) topically applying to the skin a composition comprising at least one imidopercarboxylic acid derivative,

b) leaving the composition in contact with the skin for an appropriate period for the pro-desquamating action of the said derivative, which may be between 5 min and 6 hours, preferably between 5 min and 30 min, and

c) eliminating the composition by rinsing.

According to an alternative embodiment, the invention relates to a method, in particular a cosmetic method, for promoting desquamation of the skin and/or of the mucous membranes, comprising at least one step (i) for preparing the skin for peeling consisting in applying to the regions to be treated at least one imidopercarboxylic acid derivative, at least at a suitable concentration for causing desquamation, and (ii) a subsequent step comprising the application of at least one other auxiliary desquamating agent at a concentration less than that causing the desquamation. A step of eliminating the desquamating agent(s) by rinsing will then be performed.

Advantageously, step (i) may be repeated with increasing concentrations of imidopercarboxylic acid derivative. These concentrations will be adjusted by persons skilled in the art according to the desired effect and the number of applications envisaged, but will be generally less than 50%. It will be possible, for example, to use a first concentration of about 10%, and then one or more successive applications with a concentration of about 5%, and then 5 or 3% by weight of active material relative to the total weight of the composition.

This method is more particularly intended for preventing, attenuating and/or combating the signs of skin ageing, and in particular attenuating wrinkles and fine lines, and improving the appearance and/or the texture of the skin, in particular improving the homogeneity of the complexion and/or smoothing the skin, and in particular attenuating acne or chickenpox marks, and/or clearing skin pores.

Imidopercarboxylic Acid Derivatives

According to one embodiment of the invention, the imidopercarboxylic acid derivative is represented by the compound of formula (I) which follows,

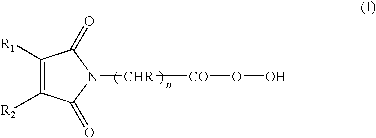

in which:

R₁ and R₂ represent independently of each other (i) a hydrogen atom, (ii) a (C₁-C₅)alkyl group optionally substituted with one or more, preferably with one to three groups chosen from a (C₁-C₅)alkoxy group, —OH group, the —COOH group, the —COOOH group, a —COOR₄ group, the —NO₂ group and a halogen atom or (iii) a (C₁-C₅)alkoxy group,

or else R₁ and R₂ form together
either a non-aromatic 5- or 6-membered ring optionally substituted with one or more, preferably with one to three groups chosen from a (C₁-C₅)alkyl group optionally substituted with one or more, preferably with one to three groups chosen from a (C₁-C₅)alkoxy group, the —OH group, the —COOH group, the —COOOH group, a —COOR₄ group, the —NO₂ group and a halogen atom or a (C₁-C₅)alkoxy group and R₄, R and n are as defined below,
or an aromatic ring A in order to form more particularly a compound derived from an aromatic imidopercarboxylic acid derivative of formula (II)

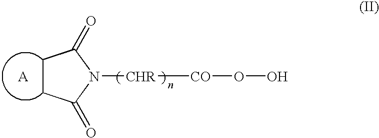

in which:

A represents a benzene or naphthalene ring optionally substituted with one or more, preferably up to 4, and more preferably still with 1 or 2 groups chosen from a radical R₃, a (C₁-C₅)alkoxy group, the —OH group, the —COOH group, the —COOOH group, a —COOR₄ group, the —NO₂ group and a halogen atom,

the R group(s), which are identical or different, represent(s) a hydrogen atom, an —OH group, a —COOH group, a —COOOH group, a —COOR₄ group or a radical R₃,

R₃ represents a (C₁-C₅)alkyl group optionally substituted with one or more, preferably with one to three groups chosen from a (C₁-C₅)alkoxy group, the —OH group, the —COOH group, the —COOOH group, a —COOR₄ group, the —NO₂ group and a halogen atom,

R₄ represents a (C₁-C₅)alkyl group optionally substituted with a group chosen from a (C₁-C₅)alkoxy group, the —OH group, the —COOH group, the —COOOH group, and a —COOR₅ group where R₅ is a (C₁-C₅)alkyl group, and

n is an integer varying from 1 to 11, in particular from 1 to 5, and for example from 1 to 3 or for example from 3 to 5.

It is understood that the compound of formula (I) or (II) comprises all the optional enantiomers and mixtures thereof and the corresponding salts or any dimer of the compound of formula (I) or (II).

Among the salts, there may be mentioned the potassium salts or the ammonium salts for example.

Examples of (C₁-C₅)alkyl groups are methyl, ethyl, n- and i-propyl, n-, i-, sec- and tert-butyl and pentyl. Among the (C₁-C₅)alkoxy groups, there may be mentioned methoxy and ethoxy. Among the halogen atoms, there may be mentioned fluorine, chlorine and bromine.

Among the substituted (C₁-C₅)alkyl groups, there may be mentioned in particular the groups —CH₂OCH₃, —CH₂OC₂H₅, —CH₂OH, —CH₂COOH, —CH₂COOOH, —CH₂CH₂COOH, —CH₂CH₂COOOH, —CH₂Cl, —CH₂F, —CF₃ and —CH₂COOC₂H₅.

According to one embodiment, the R group is a hydrogen atom.

According to another embodiment, A represents a benzene ring.

According to yet another embodiment, A is substituted with at least one —COOH group or one —COOOH group.

Finally, according to another embodiment, A is not substituted.

According to another of its aspects, the subject of the invention is more particularly the cosmetic use of at least one aromatic imidopercarboxylic acid derivative of formula (II) for which A is an unsubstituted benzene or naphthalene ring, R is a hydrogen atom, a —COOOH group or a (C₁-C₃)alkyl group optionally substituted with a —COOOH group or a —COOH group, and n varies from 3 to 5, in a composition comprising a physiologically acceptable medium, as agent intended for promoting desquamation of the skin and/or of the scalp and/or for stimulating epidermal renewal.

Among the compounds of formula (I), there may be mentioned the following compounds: 2,5-dihydro-3-methyl-2,5-dioxo-1H-pyrrole-1-hexaneperoxoic acid (R₁=H, R₂=Me; n=5); 2,5-dihydro-2,5-dioxo-1H-pyrrole-1-hexaneperoxoic acid (R₁=R₂=H; n=5), and 1,3,4,5,6,7-hexahydro-1,3-dioxo-2H-isoindole-2-butaneperoxoic acid (R₁ and R₂=6-membered ring; n=3).

Among the compounds of formula (II), there may be mentioned, by the customary names, the following compounds: phthalimidoperacetic acid (A=benzene; n=1), 3-phthalimidoperpropionic acid (A=benzene; n=2), 2-phthalimidodipersuccinic acid (A=benzene; R=COOOH; n=2), 3-phthalimidoperbutyric acid (A=benzene; R=CH₃; n=2), 2-phthalimidoperpropionic acid (A=benzene; R=CH₃; n=1), 2-phthalimidomono-perglutaric acid methyl semi-ester (A=benzene; R=—COOOMe; n=3), 3-phthalimidodiperadipic acid (A=benzene; R=COOOH; n=4), naphthalimidoperacetic acid (A=naphthalene; n=1), 2-phthalimidomonopersuccinic acid (A=benzene; R=COOH; n=2) and 4-(4-percarboxy)phthalimidoperbutyric acid.

There may also be mentioned, among the compounds of formula (II), the following compounds, cited by CAS names:
compounds for which A is a benzene ring, for example 1,3-dihydro-1,3-dioxo-2H-isoindole-2-butaneperoxoic acid, also called phthalimidoperoxybutanoic acid, 3-phthalimidoperoxybutanoic acid or 4-phthalimidoperbutyric acid (n=3), 1,3-dihydro-1,3-dioxo-2H-isoindole-2-pentaneperoxoic acid (n=4), 1,3-dihydro-1,3-dioxo-2H-isoindole-2-heptaneperoxoic acid (n=6), 1,3-dihydro-1,3-dioxo-2H-isoindole-2-octaneperoxoic acid (n=7) or phthalimidoperoctanoic acid, 1,3-dihydro-1,3-dioxo-2H-isoindole-2-nonaneperoxoic acid (n=8), 1,3-dihydro-1,3-dioxo-2H-isoindole-2-decaneperoxoic acid (n=9), 1,3-dihydro-1,3-dioxo-2H-isoindole-2-undecaneperoxoic acid (n=10), 1,3-dihydro-1,3-dioxo-2H-isoindole-2-dodecaneperoxoic acid (n=11);
compounds for which A is a substituted benzene ring, for example 1,3-dihydro-5-(hydroperoxycarbonyl)-1,3-dioxo-2H-isoindole-2-butaneperoxoic acid (n=3, substitution with a —COOOH group), 1,3-dihydro-4-(hydroperoxycarbonyl)-1,3-dioxo-2H-isoindole-2-butaneperoxoic acid (n=3, substitution with a —COOOH group);
compounds for which A is a benzene ring and at least one of the R groups is different from a hydrogen atom, for example 3-(1,3-dihydro-1,3-dioxo-2H-isoindol-2-yl)hexanediperoxoic acid (n=3, R=CH₂COOOH), (1,3-dihydro-1,3-dioxo-2H-isoindol-2-yl)butanediperoxoic acid (R=COOOH, n=2), 2-(1,3-dihydro-1,3-dioxo-2H-isoindol-2-yl)pentanediperoxoic acid (R=COOOH, n=3), 5-methyl ester of 4-phthalimidomono-peroxyglutaric acid, (R=COOMe, n=3), 1,3-dihydro-β-methyl-1,3-dioxo-2H-isoindole-2-propaneperoxoic acid also called β-methyl-1,3-dioxoperoxy-2-isoindolinepropionic acid or 3-phthalimidoperbutyric acid (R=Me, n=2).

The following salt may also be mentioned: N-(2-hydroxyethyl)-N,N-dimethyl-1-dodecanaminium salt of 1,3-dihydro-1,3-dioxo-2H-isoindole-2-hexaneperoxoic acid (1:1).

According to yet another embodiment, the compound of formula (II) is phthalimidoperoxycaproic acid, also called 6-(phthalamidoperoxy)hexanoic acid, e-(phthalimidoperoxy)hexanoic acid or PAP. Its chemical structure is as follows:

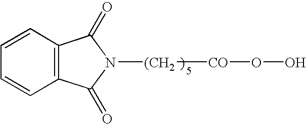

It corresponds to a compound of formula (II) in which the R group represents a hydrogen atom, A represents an unsubstituted benzene ring and n=5.

Finally, by way of an acceptable dimer of a compound of formula (II), there may be mentioned the PAP dimer also called 5,7-dihydro-1,3,5,7-tetraoxobenzo[1,2-c:4,5-c′]dipyrrole-2,6(1H,3H)-dihexaneperoxoic acid of formula

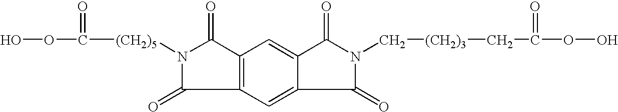

The quantity of compound of formula (I) or (II) can be used according to the invention, of course derived from the desired effect, should be an effective quantity for promoting desquamation of the skin and/or of the scalp and/or for stimulating epidermal renewal.

By way of example, the quantity of imidopercarboxylic acid derivative, in particular of compound of formula (I) or (II), that can be used according to the invention may range for example from 0.1 to 50%, in particular from 1 to 50%, in particular from 0.1 to 20%, in particular from 1 to 20%, and preferably from 0.1 to 10%, in particular from 1 to 10% by weight of active material, relative to the total weight of the composition.

For conventional applications, 0.1 to 10% by weight of active material will be used relative to the total weight of the composition, in particular 0.2 to 5% by weight, and preferably 0.2 to 2% by weight of active material, relative to the total weight of the said composition.

For peeling applications, an amount greater than 10% by weight of active material will be preferably used relative to the total weight of the composition, in particular ranging from 11% to 50% by weight of active material, in particular from 11 to 40% and more preferably still from 11 to 30% by weight of active material, relative to the total weight of the composition.

The phthalamidoperoxycaproic or phthalamidoperoxyhexanoic acid (PAP) is available in particular in two commercial forms by the company SOLVAY.

Thus, phthalamidoperoxycaproic acid may be used together with cyclodextrins so as to improve the stability of the compound.

The document EP 895 777, already cited in the preamble, describes more particularly the formation of an adduct of e-phthalamidoperoxyhexanoic acid with cyclodextrin.

Such an encapsulated product is sold by the company SOLVAY under the trade name EURECO® HC-P11 (Powdered inclusion complex of Phthalimidoperoxycaproic acid with pharmagrade β-cyclodextrin).

The phthalamidoperoxycaproic acid is also supplied in the form of an aqueous dispersion (containing 17% of active material) under the trade name EURECO® HC L17.

The phthalamidoperoxycaproic acid is also supplied in the form of an aqueous dispersion (containing 5% of active material) under the trade name EURECO® HC L5.

The phthalamidoperoxycaproic acid is also supplied in the form of a powder under the trade name EURECO® HC-P11 (containing 11% of active material).

Such aqueous dispersions of phthalamidoperoxycaproic acid are described in the document EP 1 074 607. These aqueous dispersions are in reality stabilized by copolymers of methyl vinyl ether with maleic acid and/or anhydride, in a 1:1 ratio, having an alternating structure.

According to a preferred particular mode, phthalamidoperoxycaproic acid non-complexed with cyclodextrin will be used in powdered form or in the form of an aqueous dispersion. A preferred form is an aqueous dispersion.

Other galenic forms comprising an imidopercarboxylic acid derivative may also be suitable for carrying out the present invention. In this regard, there may be mentioned in particular dispersions, in particular aqueous dispersions, as described in patent application WO 2004/110610, capsules comprising a gel-based matrix as described in patent application WO 2004/110611, capsules based on an inorganic salt as described in application WO 2004/110612 or multilayer capsules comprising at least one polyelectrolyte and/or one ionic surfactant as described in patent application WO 2004/110613.

Auxiliary Desquamating Agent

According to the present invention, the compound of formula (I) may be used in combination with an auxiliary desquamating agent, in particular chosen from beta-hydroxy acids such as salicylic acid and its derivatives, in particular 5-n-octanoylsalicylic acid, urea and its derivatives chosen from a hydroxyethylurea, in particular N-(2-hydroxyethyl)urea marketed under the name Hydrovance® by National Starch, a thiourea, N-2-hydroxyethylpiperazine-N-2-ethanesulphonic acid (HEPES), Sophora japonica extract, honey, N-acetylglucosamine, 2-oxothiazolidine-4-carboxylic acid (procysteine) derivatives, extracts of laminaria saccharina and laminaria ochroleuca and mixtures thereof.

Thus, this object of the present invention is also a care composition for the skin and/or the scalp, comprising the combination of a derivative of formula (I) or (II) and an auxiliary desquamating agent, characterized in that the auxiliary desquamating agent is chosen from beta-hydroxy acids, such as salicylic acid and its derivatives, in particular 5-n-octanoylsalicylic acid; urea and its derivatives chosen from hydroxyethylurea, in particular N-(2-hydroxyethyl)urea marketed under the name Hydrovance® by National Starch, thiourea; (N-2-hydroxyethylpiperazine-N-2-ethane)sulphonic acid (HEPES); Sophora japonica extract; honey; N-acetylglucosamine, 2-oxothiazolidine-4-carboxylic acid (procysteine) derivatives, extracts of laminaria such as laminaria saccharina and laminaria ochroleuca and mixtures thereof.

According to a preferred embodiment, the composition will contain at least salicylic acid and/or one of its derivatives, in particular 5-n-octanoylsalicylic acid. According to a particular embodiment, the said composition will contain 5-n-octanoylsalicylic acid. According to another particular embodiment, the composition will contain 5-n-octanoylsalicylic acid and salicylic acid.

In addition to the abovementioned auxiliary desquamating agent, the composition may also contain another desquamating agent, called hereinafter “additional desquamating agent” belonging to the general list of desquamating agents proposed below.

The expression “desquamating agent” is understood to mean any compound capable of acting:
either directly on desquamation by promoting exfoliation, such as: saturated and unsaturated monocarboxylic acids, saturated and unsaturated dicarboxylic acids, saturated and unsaturated tricarboxylic acids; alpha hydroxy acids and beta hydroxy acids of monocarboxylic acids; alpha-hydroxy acids and beta-hydroxy acids of dicarboxylic acids; alpha-hydroxy acids and beta-hydroxy acids of tricarboxylic acids, keto acids, alpha-keto acids, beta-keto acids of polycarboxylic acids, polyhydroxy monocarboxylic acids, polyhydroxy bicarboxylic acids, polyhydroxy tricarboxylic acids; and (3-hydroxy-2-pentylcyclopentyl)acetic acid.

As preferred α-hydroxy acids, there may be mentioned: glycolic acid, citric acid, lactic acid, tartric acid, malic acid or mandelic acid.

The preferred β-hydroxy acids are chosen from: salicylic acid and its derivatives, in particular 5-n-octanoylsalicylic acid.

As other desquamating agents, there may be mentioned: pyruvic, gluconic, glucuronic, oxalic, malonic, succinic, acetic, gentisic, cinnamic and azelaic acids; phenol, resorcinol; oligofucoses as in patent EP 0 218 200; jasmonic acid and its derivatives as in patent applications EP 1 333 022 and EP 1 333 021; trichloroacetic acid; retinoids such as retinol, retinoic acid; adapalene; resveratrol; and their salts and derivatives, such as their cis or trans forms, the racemic mixtures, the dextrorotatory or levorotatory forms of the abovementioned agents.
or on the enzymes involved in desquamation or the degradation of the corneodesmosomes, such as glycosidases, stratum corneum chymotryptic enzyme (SCCE) or even other proteases (trypsin, chymotrypsin-like).

It is also possible to mention in particular the derivatives of glycine-type alpha-amino acids (as described in EP 0 852 949, as well as sodium methylglycine diacetate marketed by BASF under the trade name TRILON M®); sugar derivatives such as O-octanoyl-6-D-maltose, O-linoleyl-6-D-glucose and N-acetylglucosamine.

As additional desquamating agent, there may also be mentioned glycerol trilactate, silicon derivatives of salicylate as in patent EP 0 796 861, salts of 5-acylsalicylic acid, active agents having effects on transglutaminase as in patent EP 0 899 330, and a flower extract of the ficus Opuntia indica such as Exfolactive® from Silab.

According to a particular embodiment, the auxiliary desquamating agent is chosen from 5-n-octanoylsalicylic acid, (N-2-hydroxyethylpiperazine-N-2-ethane)-sulphonic acid (HEPES), derivatives of 2-oxothiazolidine-4-carboxylic acid (procysteine) and mixtures thereof. Preferably still, 5-n-octanoylsalicylic acid will be used. Persons skilled in the art will know how to define the required quantity of total desquamating agent, namely of auxiliary desquamating agent and additional desquamating agent optionally present in combination in order to obtain the desired effect on the skin and/or the scalp.

By way of example, the auxiliary desquamating agent and the additional desquamating agent may be present in an amount ranging from 0.001 to 95% by weight relative to the total weight of the composition, in particular from 0.01 to 30% by weight relative to the total weight of the composition and preferably from 0.01 to 10% by weight relative to the total weight of the composition.

When the composition used according to the invention has an application in peeling, it may in particular comprise from 0.01 to 90% by weight of desquamating agent(s) in their entirety relative to the total weight of the composition, preferably from 1 to 70% by weight relative to the total weight of the composition.

Preferably, there will be used in the peeling compositions at least 10% by weight, and in particular more than 20% by weight, in particular from 21 to 50%, and preferably from 21 to 30% of auxiliary desquamating agent and additional desquamating agent relative to the total weight of the composition.

Additional Agent

The composition which can be used according to the present invention may additionally comprise at least one cosmetic and/or dermatological additional agent intended for improving the appearance and/or the comfort of the skin and/or of the scalp.

This additional agent may be in particular an anti-ageing and/or anti-wrinkle agent in particular chosen from an antiglycation agent, an NO-synthase inhibitor, an agent acting on the dermal or epidermal macromolecules and/or preventing their degradation, an agent modulating the differentiation or the proliferation of the cells, and in particular the differentiation of the keratinocytes and/or the proliferation of the fibroblasts or of the keratinocytes, a muscle relaxant and/or skin relaxant.

The additional agent may also be a moisturizing agent, a soothing agent or a mixture.

Accordingly, the anti-ageing and/or anti-wrinkle agent may in particular be chosen from:
an antiglycation agent, such as an extract of bilberry (Vaccinium myrtillus), for example that sold under the name “BLUEBERRY HERBASOL EXTRACT PG” by the company COSMETOCHEM, a black tea extract such as Kombuchka from Sederma, or lipoic or thioctic acid such as Nutralip® from Labochim;
an NO-synthase inhibitor, such as extracts of Gingko biloba, of Vitis vinifera or of Olea europaea;
an agent acting on dermal or epidermal macromolecules and/or preventing their degradation, such as Centella asiatica extracts, asiaticosides and derivatives; ascorbic acid and its derivatives; synthetic peptides such as iamine, biopeptide CL or palmitoyl pentapeptide marketed by the company SEDERMA under the trade name Matrixyl®; malt extract marketed by the company COLETICA under the trade name Collalift®; lycopene; Saccharomyces cerevisiae extract available in particular from the company SILAB under the trade name Firmalift® or from the company LS under the trade name Cytovitin®; or on the inhibition of the degradation of elastin such as the peptide extract of Pisum sativum seeds marketed by the company LS under the trade name Parelastyl®; the N-acylamino amide compounds described in application WO 01/94381 such as {2-[acetyl-(3-trifluoromethylphenyl)amino]-3-methylbutyrylamino}acetic acid, otherwise called N—[N-acetyl,N′-(3-trifluoromethyl)phenylvalyl]glycine or N-acetyl-N-[3-(trifluoromethyl)phenyl]valylglycine or acetyl trifluoromethyl phenyl valylglycine, or an ester thereof with a C₁-C₆ alcohol; an extract of rice peptides such as Colhibin® from Pentapharm, the extract of brown alga Padina pavonica marketed by the company ALBAN MÜLLER under the trade name HPS3®; a Laminaria ochroleuca extract such as Laminaine® from BIOTECHMARINE; the lupin extract marketed by the company SILAB under the trade name Structurine®; the beech Fagus sylvatica bud extract marketed by the company GATTEFOSSE under the trade name Gatuline® RC; a peptide extract of Voandzeia substerranea such as that marketed by the company LS under the trade name Filladyn LS 9397®; niacinamide or vitamin B3; in particular, Centella asiatica extracts will be used;
an agent modulating the differentiation or the proliferation of the cells, and in particular the differentiation of the keratinocytes and/or the proliferation of the fibroblasts or of the keratinocytes and such as a soybean extract (hydrolysed proteins of soybean) such as RIDULISSE® C from SILAB, 1,3,5-trimethoxybenzene (phloroglucinol), a lactobacillus filtrate, Phosphovital® from Sederma, Stimoderm® from CLR, Vigna aconitifolia seeds, Vitoptine® from LS, hazelnut peptides, Nuteline® from Solabia and the lucerne extract Vitanol from SILAB;
a muscle relaxant and/or skin relaxant, such as magnesium gluconate, manganese gluconate and other salts, alverine citrate and its salts, glycine, an Iris pallida extract, a hexapeptide (Argireline R from Lipotec) or sapogenins such as wild yam and the carbonyl amines described in application EP1 484 052, and adenosine.

These additional anti-ageing agents are generally present in the cosmetic compositions which can be used according to the present invention in amounts ranging from 0.01 to 20% by weight relative to the total weight of the composition, preferably from 0.01 to 10% by weight relative to the total weight of the composition and more preferably still from 0.01 to 1% by weight relative to the total weight of the composition.

The invention also relates to a cosmetic and/or dermatological composition comprising:
at least one imidopercarboxylic acid derivative, in particular a compound of formula (I) or (II), and
at least one auxiliary desquamating agent as detailed above for example chosen from 5-n-octanoylsalicylic acid, (N-2-hydroxyethylpiperazine-N-2-ethane)-sulphonic acid (HEPES), and mixtures thereof.

The invention also relates to a cosmetic and/or dermatological composition comprising:
at least one imidopercarboxylic acid derivative, in particular a compound of formula (I) or (II), and
at least one additional anti-ageing agent, and preferably at least two anti-ageing agents chosen from adenosine, a malt extract, a Padina pavonica extract, a Centella asiatica extract, a soybean extract, a wild yam extract, an agent modulating the differentiation or the proliferation of cells, and in particular the differentiation of the keratinocytes and/or proliferation of the fibroblasts or of the keratinocytes, as defined above and mixtures thereof
and optionally a moisturizing agent and/or a soothing agent.

For the treatment of dry skin, it will be advantageous to use, as additional agent in the composition of the invention, moisturizing agents, soothing agents and mixtures thereof.

As moisturizing agents, there may be mentioned in particular hyaluronic acid, homopolymers of acrylic acid such as Lipidure-HM® from NOF Corporation, beta-glucan and in particular sodium carboxymethyl-beta-glucan from Mibelle-AG-Biochemistry; a mixture of passionflower, apricot, corn and rice bran oils marketed by Nestlé under the name NutraLipids®; a C-glycoside derivative such as those described in application WO 02/051828 and in particular C-β-D-xylopyranoside-2-hydroxypropane in the form of a solution containing 30% by weight of active material in a water/propylene glycol mixture (60/40% by weight) such as the product manufactured by CHIMEX under the trade name “MEXORYL SBB®”; a musk rose oil marketed by Nestlé; a zinc-enriched extract of the microalga prophyridium cruentrum marketed by Vincience under the name Algualane Zinc®; spheres of collagen and of chondroitin sulphate of marine origin (Ateocollagen) marketed by the company Engelhard Lyon under the name “sphères de comblement marines”; hyaluronic acid spheres such as those marketed by the company Engelhard Lyon; and arginine.

As soothing and/or anti-irritant agent which makes it possible to reduce the sensation of tingling, itching or tightness of the skin, there may be mentioned in particular β-glycyrrhetinic acid and its salts or derivatives (stearyl glycyrrhetate, 3-stearoyloxyglycyrrhetic acid, glycyrrhetinic acid mono glucuronide) and plants containing them (for example: Glycyrrhiza glabra); ursolic acid and its salts; Centella asiatica extracts, Canola oil, bisabolol; camomile extracts, allantoin; a mixture of water lily flower extract and palmitoylproline such as that sold under the name “Seppicalm VG®” by the company Seppic; Aloe vera, rose water, an extract of mint, in particular of mint leaves such as Calmiskin® from Silab, filamentous bacteria such as Vitreoscilla filiformis as described in patent EP 761 204 and marketed by Chimex under the name Mexoryl SBG®, an extract of rose petals such as Rose Flower Herbasol® extract from the company Cosmetochem, shea butter, a fermented extract of Alteromonas marketed under the name ABYSSINE® by the company Atrium Biotechnologies; thermal springs of the Vichy basin, such as water obtained from the Célestins, Chomel, Grande-Grille, Hôpital, Lucas and Parc springs, and preferably water from the Lucas spring; an extract of Eperua falcata bark such as that marketed by the company COGNIS under the name Eperuline®; an extract of Paeonia suffructicosa root such as that marketed by the company Ichimaru Pharcos under the name Botanpi Liquid B®; and mixtures thereof.

The invention also relates to a cosmetic and/or dermatological composition comprising:
at least one imidopercarboxylic acid derivative, in particular a compound of formula (I) or (II), and
at least one additional agent chosen from a moisturizing agent, a soothing agent and mixtures thereof, and preferably at least two agents chosen from hyaluronic acid, a mixture of passionflower, apricot, corn and rice bran oils marketed by Nestlé under the name NutraLipids®; a C-glycoside derivative such as those described in application WO 02/051828 and in particular C-β-D-xylopyranoside-2-hydroxypropane in the form of a solution containing 30% by weight of active material in a water/propylene glycol mixture (60/40% by weight) such as the product manufactured by CHIMEX under the trade name “MEXORYL SBB®”; a musk rose oil marketed by Nestlé; spheres of collagen and of chondroitin sulphate of marine origin (Ateocollagen) marketed by the company Engelhard Lyon under the name “sphères de comblement marines”; hyaluronic acid spheres such as those marketed by the company Engelhard Lyon; Centella asiatica extracts, allantoin; Aloe vera, rose water, an extract of mint, in particular of mint leaves such as Calmiskin® from Silab, filamentous bacteria such as Vitreoscilla filiformis as described in patent EP 761 204 and marketed by Chimex under the name Mexoryl SBG®, an extract of rose petals such as Rose Flower Herbasol® extract from the company Cosmetochem, shea butter, thermal springs of the Vichy basin, such as water obtained from the Célestins, Chomel, Grande-Grille, Hôpital, Lucas and Parc springs, and preferably water from the Lucas spring; and mixtures thereof.

The compositions which can be used according to the invention comprise a physiologically acceptable medium.

The expression “physiologically acceptable medium” is understood to mean a medium compatible with the keratin fibres and/or materials of human beings, such as for example, without limitation, the skin, the mucous membranes, the nails, the scalp and/or the hair.

This physiologically acceptable medium comprises water, optionally mixed or not mixed with one or more organic solvents such as C₁-C₈ alcohols, in particular ethanol, isopropanol, tert-butanol, n-butanol, polyols such as glycerine, propylene glycol, butylene glycol and polyol ethers.

The compositions which can be used according to the invention may be a cosmetic or dermatological composition and may therefore comprise a cosmetically or pharmaceutically acceptable medium.

The composition may also comprise a fatty phase, which may comprise oils, gums, waxes customarily used in the field of application considered. As oils or waxes which can be used in the invention, there may be mentioned mineral oils (liquid paraffin), vegetable oils (liquid fraction of shea butter, sunflower oil, apricot oil, rice bran oil and the like), animal oils (perhydrosqualene) synthetic oils (purcellin oil), silicone oils or waxes (cyclomethicone, dimethicone) and fluorinated oils (perfluoropolyethers), beeswax, carnauba wax or paraffin wax, shea butter, hydrogenated jojoba oil. Fatty alcohols (cetyl alcohol, stearyl alcohol and the like) and fatty acids (stearic acid and the like) may be added to these oils.

When the composition is an emulsion, the proportion of the fatty phase may range from 5% to 80% by weight, and preferably from 5% to 50% by weight relative to the total weight of the composition. The oils, waxes, emulsifiers and coemulsifiers used in the composition in the form of an emulsion are chosen from those conventionally used in the cosmetic field. The emulsifier and the coemulsifier are present in the composition in a proportion ranging from 0.3% to 30% by weight, and preferably from 0.5 to 20% by weight relative to the total weight of the composition. The emulsion may additionally contain lipid vesicles.

When the composition is a solution or an oily gel, the fatty phase may represent more than 90% of the total weight of the composition.

The composition may also contain the usual adjuvants in the field considered, such as surfactants, emulsifiers, hydrophilic or lipophilic gelling agents, hydrophilic or lipophilic additives, preservatives, antioxidants, solvents, perfumes, fillers, screening agents, odour absorbers and colouring matter, other cosmetic or pharmaceutical active agents. The quantities of these various adjuvants are those conventionally used in the cosmetic field, and are for example from 0.01% to 10% of the total weight of the composition. These adjuvants, depending on their nature, may be introduced into the fatty phase, into the aqueous phase and/or into the lipid spherules.

As surfactants which are capable of being used, there may be mentioned for example glycerol stearate, polysorbate 60 and the PEG-6/PEG-32/glycol stearate mixture sold under the name TefoseR 63 by the company Gattefosse; PEG stearate derivatives, sugar derivatives.

As hydrophilic gelling agents which can be used in the invention, there may be mentioned carboxyvinyl polymers (carbomer), acrylic copolymers such as acrylate/alkyl acrylate copolymers, polyacrylamides, polysaccharides such as hydroxypropylcellulose, natural gums and clays, and, as lipophilic gelling agents, there may be mentioned modified clays such as bentones, metal salts of fatty acids such as aluminium stearates and hydrophobic silica, ethyl cellulose, polyethylene.

The composition may be provided in all the conceivable galenic forms.

In particular, the composition may be in the form of an aqueous, alcoholic, aqueous-alcoholic or oily solution; a dispersion of the lotion or serum type; a water-in-oil, oil-in-water or multiple emulsion; a suspension; microcapsules or microparticles; vesicular dispersions of the ionic and/or non-ionic type; an aqueous or oily lotion or in serum form; a foam, a solid preparation, for example a stick; an aerosol composition also comprising a propellant under pressure or in the form of a patch.

The composition which can be used according to the invention may be provided in the form of a composition for hair care, in particular a shampoo, a hair setting lotion, a treatment lotion, a hair styling cream or gel, a dye composition, in particular an oxidation dye, composition, restructuring lotions for the hair, a permanent-waving composition (in particular a composition for the first stage of a permanent waving), an anti-hair-loss lotion or gel, an antiparasitic shampoo.

It may also be provided in the form of a cleansing, protective, treatment or care composition for the face, for the hands, for the feet, for the large anatomical folds or for the body (for example day creams, night cream, makeup-removing cream, anti-sun composition, protection or care body milks, after-sun milks, care lotion, gel or foam for the skin, such as cleansing lotion, artificial tanning composition); a make-up composition for the body or the face such as a foundation; a bath composition; a deodorant composition comprising for example a bactericidal agent; an aftershave composition; a depilatory composition; a composition for insect bites; an anti-pain composition; a composition for treating certain skin diseases such as eczema, rosacea, psoriasis, lichens and severe pruritus.

When the composition which can be used according to the invention is intended for a peeling-type use, it may also be provided in all the galenic forms mentioned above as long as it can be easily removed by rinsing, and in particular in the form of an aqueous gel, an aqueous or aqueous-alcoholic solution. It may be applied by any means allowing uniform distribution and in particular with the aid of cotton wool, a wand, a brush, a gauze, a spatula or a pad, or else by spraying, and may be removed by rinsing with water or with the aid of a mild detergent. According to a preferred embodiment of the invention, the composition intended for chemical peeling contains a continuous aqueous phase.

The following examples illustrate the present invention.

### EXAMPLE 1

### Test of Desquamating Efficacy

An in vitro test was carried out in order to determine the efficacy of phthalimidoperoxycaproic acid compared with a composition comprising 30% of glycolic acid and 70% of water. The number of corneocytes liberated is expressed as protein concentration. After applying a composition comprising 17% of phthalimidoperoxycaproic acid, the protein concentration is about 320 μg/ml, while it is about 210 μg/ml for the composition comprising glycolic acid.

This test shows that phthalimidoperoxycaproic acid (PAP) has a high desquamating efficacy greater than the reference glycolic acid (AG) at 30%.

Unless otherwise stated, the % values indicated in the compositions below are expressed as weight of raw material.

### EXAMPLE 2

### Anti-Ageing Composition

| | % by weight |
|---|---|
| Phthalimidoperoxycaproic acid⁽¹⁾ | 10 |
| Dimethyl isosorbide | 10 |
| Glycerine | 3 |
| Alcohol | 10 |
| Water | qs 100 |

⁽¹⁾Eureco ® HCL17 (17% by weight of active material) sold by the company SOLVAY.

### EXAMPLE 3

### Anti-Ageing Composition

| | % by weight |
|---|---|
| Phthalimidoperoxycaproic acid⁽¹⁾ | 10 |
| Hydroxypropyl cellulose | 0.5 |
| Alcohol | 10 |
| Water | qs 100 |

⁽¹⁾Eureco ® HCL17 (17% by weight of active material) sold by the company SOLVAY.

### EXAMPLE 4

### Composition for Dry Skin

| | % by weight |
|---|---|
| Phthalimidoperoxycaproic acid⁽¹⁾ | 5 |
| 5-n-Octanoylsalicylic acid | 0.5 |
| Centella asiatica extract | 0.01 |
| Mixture of passion flower, apricot, corn and rice bran oils⁽²⁾ | 1 |
| Surfactants | 2 |
| Glycerol | 5 |
| Water | qs 100 |

⁽¹⁾Eureco ® HCL17 (17% by weight of active material) sold by the company SOLVAY.
⁽²⁾Nutralipids ® sold by Nestle.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-15. (canceled)

16. A method of cosmetic treatment for non-therapeutic care of the skin and/or the scalp, comprising:
applying a composition comprising at least one imidoperoxycarboxylic acid derivative to the skin and/or the scalp;
thereby
promoting desquamation of the skin, the scalp or both the skin and the scalp,
stimulating epidermal renewal, or
both promoting desquamation of the skin, the scalp and both the skin and the scalp and stimulating epidermal renewal.

17. The method according to claim 16, wherein the application of the composition comprising at least one imidoperoxycarboxylic acid derivative promotes at least one effect selected from the group consisting of preventing, attenuating and/or combating the signs of skin ageing, improving the appearance and/or the texture of the skin and/or of the scalp, improving the radiance and the homogeneity of the complexion and/or smoothing the skin, reducing the surface irregularities and the skin microrelief, promoting the elimination of dead cells at the surface of the body, promoting the elimination of dandruff, improving the staying power of the makeup, and improving the result of treatment of the skin with colorants for the stratum corneum.

18. The method according to claim 16, wherein the at least one imidoperoxycarboxylic acid derivative is an aromatic imidopercarboxylic acid derivative of formula (I) 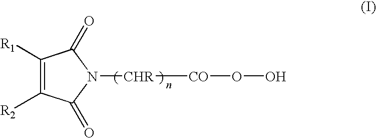
wherein:
R₁ and R₂ represent independently of each other (i) a hydrogen atom, (ii) a (C₁-C₅)alkyl group optionally substituted with one to three groups selected from the group consisting of a (C₁-C₅)alkoxy group, a —OH group, a —COOH group, a —COOOH group, a —COOR₄ group, a —NO₂ group and a halogen atom or (iii) a (C₁-C₅)alkoxy group,
or R₁ and R₂ form
either a non-aromatic 5- or 6-membered ring optionally substituted with one to three groups selected from the group consisting of a (C₁-C₅)alkyl group optionally substituted with one to three groups selected from the group consisting of a (C₁-C₅)alkoxy group, a —OH group, a —COOH group, a —COOOH group, a —COOR₄ group, a —NO₂ group, a halogen atom and a (C₁-C₅)alkoxy group,
wherein R₄, R and n are as defined below, or
an aromatic ring A in order to form a compound derived from an aromatic imidopercarboxylic acid derivative of formula (II) 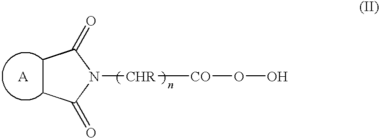
wherein:
A represents a benzene or naphthalene ring optionally substituted with one to 4 groups selected from the group consisting of a radical R₃, a (C₁-C₅)alkoxy group, a —OH group, a —COOH group, a —COOOH group, a —COOR₄ group, a —NO₂ group and a halogen atom,
the R group(s), which are identical or different, represent(s) a hydrogen atom, an —OH group, a —COOH group, a —COOOH group, a —COOR₄ group or a radical R₃,
R₃ represents a (C₁-C₅)alkyl group optionally substituted with one to three groups selected from the group consisting of a (C₁-C₅)alkoxy group, a —OH group, a —COOH group, a —COOOH group, a —COOR₄ group, a —NO₂ group and a halogen atom,
R₄ represents a (C₁-C₅)alkyl group optionally substituted with a group selected from the group consisting of a (C₁-C₅)alkoxy group, a —OH group, a —COOH group, a —COOOH group, and a —COOR₅ group
wherein R₅ is a (C₁-C₅)alkyl group, and
n is an integer from 1 to 5.

19. The method according to claim 18, wherein the at least one imidopercarboxylic acid derivative is an aromatic imidopercarboxylic acid derivative of formula (II),
wherein
A is an unsubstituted benzene or naphthalene ring,
R is a hydrogen atom, a —COOOH group or a (C₁-C₃)alkyl group optionally substituted with a —COOOH group or a —COOH group, and
n varies from 3 to 5.

20. The method according to claim 18, wherein R is a hydrogen atom.

21. The method according to claim 18, wherein A is a benzene ring.

22. The method according to claim 16, wherein the at least one imidopercarboxylic acid derivative is phthalimidoperoxycaproic acid.

23. The method according to claim 16, wherein a quantity of the at least one imidopercarboxylic acid derivative in the applied composition ranges from 0.1 to 50%, by weight relative to the total weight of the composition.

24. The method according to claim 16, wherein a quantity of the at least one imidopercarboxylic acid derivative in the applied composition ranges from 1 to 10%, by weight relative to the total weight of the composition.

25. The method according to claim 16, wherein the applied composition further comprises an auxiliary desquamating agent.

26. A method of treatment of the skin and/or scalp, comprising:
applying a composition comprising at least one imidopercarboxylic acid derivative to the skin and/or scalp,
wherein the treatment is directed to pathologies linked to a production of a thickened horny layer and/or linked to abnormal desquamation of the skin and/or scalp.

27. The method of treatment according to claim 26, wherein the skin and/or scalp pathologies linked to a production of thickened horny layer and/or linked to abnormal desquamation is selected from the group consisting of xerosis, hyperkeratosis, ichthyosis, psoriasis, atopic dermatitis, callosities and hyperkeratosis of dry feet.

28. The method according to claim 26, wherein the at least one imidopercarboxylic acid is an aromatic imidopercarboxylic acid derivative of formula (I) 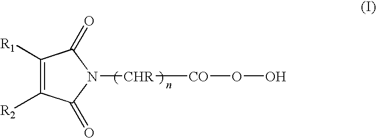
wherein:
R₁ and R₂ represent independently of each other (i) a hydrogen atom, (ii) a (C₁-C₅)alkyl group optionally substituted with one to three groups selected from the group consisting of a (C₁-C₅)alkoxy group, a —OH group, a —COOH group, a —COOOH group, a —COOR₄ group, a —NO₂ group and a halogen atom or (iii) a (C₁-C₅)alkoxy group, or
R₁ and R₂ form
either a non-aromatic 5- or 6-membered ring optionally substituted with one to three groups selected from the group consisting of a (C₁-C₅)alkyl group optionally substituted with one to three groups selected from the group consisting of a (C₁-C₅)alkoxy group, a —OH group, a —COOH group, a-COOOH group, a —COOR₄ group, a —NO₂ group and a halogen atom or a (C₁-C₅)alkoxy group and
R₄, R and n are as defined below,
or an aromatic ring A in order to form a compound derived from an aromatic imidopercarboxylic acid derivative of formula (II) 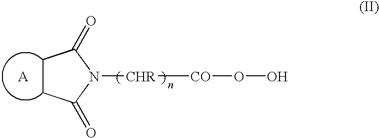
wherein:
A represents a benzene or naphthalene ring optionally substituted with one to 4 groups selected from the group consisting of a radical R₃, a (C₁-C₅)alkoxy group, a —OH group, a —COOH group, a —COOOH group, a —COOR₄ group, a —NO₂ group and a halogen atom,
the R group(s), which are identical or different, represent(s) a hydrogen atom, an —OH group, a —COOH group, a —COOOH group, a —COOR₄ group or a radical R₃,
R₃ represents a (C₁-C₅)alkyl group optionally substituted with one to three groups selected from the group consisting of a (C₁-C₅)alkoxy group, a —OH group, a —COOH group, a —COOOH group, a —COOR₄ group, a —NO₂ group and a halogen atom,
R₄ represents a (C₁-C₅)alkyl group optionally substituted with a group selected from the group consisting of a (C₁-C₅)alkoxy group, a —OH group, a —COOH group, a —COOOH group, and a COOR₅ group,
wherein R₅ is a (C₁-C₅)alkyl group, and
n is an integer from 1 to 5.

29. The method according to claim 28, wherein the imidopercarboxylic acid derivative is an aromatic imidopercarboxylic acid derivative of formula (II), wherein
A is an unsubstituted benzene or naphthalene ring,
R is a hydrogen atom, a —COOOH group or a (C₁-C₃)alkyl group optionally substituted with a —COOOH group or a —COOH group, and
n varies from 3 to 5.

30. The method according to claim 28, wherein R is a hydrogen atom.

31. The method according to claim 28, wherein A is a benzene ring.

32. The method according to claim 28, wherein the at least one imidopercarboxylic acid derivative is phthalimidoperoxycaproic acid.

33. A method for the cosmetic treatment of the visible and/or tactile irregularities of the human skin, comprising:
a) topically applying to the skin a composition comprising at least one imidopercarboxylic acid derivative;
b) leaving the composition in contact with the skin for an appropriate period for the pro-desquamating action of the at least one imidopercarboxylic acid derivative; and
c) eliminating the composition by rinsing;
wherein the appropriate period for the pro-desquamating action is between 5-min and 6 hours.

34. The method according to claim 33, wherein the treatment of the visible and/or tactile irregularities of the human skin is directed to at least one effect selected from the group consisting of attenuating and/or combating the signs of skin ageing, attenuating wrinkles and fine lines, improving the appearance and/or the texture of the skin, improving the homogeneity of the complexion, smoothing the skin, attenuating acne or chickenpox marks and clearing the skin pores.

35. A cosmetic and/or dermatological composition, comprising:
a physiologically acceptable medium;
at least one imidopercarboxylic acid derivative of formula (I) or (II) as defined according to claim 18, and
at least one auxiliary desquamating agent selected from the group consisting of a beta-hydroxy acid, urea, a hydroxyethylurea, a thiourea, N-2-hydroxyethylpiperazine-N-2-ethanesulphonic acid (HEPES), Sophora japonica extract, honey, N-acetylglucosamine, 2-oxothiazolidine-4-carboxylic acid (procysteine) derivatives, extracts of laminaria and mixtures thereof.

36. A cosmetic and/or dermatological composition, comprising:
a physiologically acceptable medium,
at least one imidopercarboxylic acid derivative of formula (I) or (II) according to claim 18, and
at least one anti-ageing agent selected from the group consisting of an antiglycation agent, an NO-synthase inhibitor, an agent acting on dermal or epidermal macromolecules and/or preventing their degradation, an agent modulating the differentiation or the proliferation of cells, a muscle relaxant and a skin relaxant.
